(19) United States
(12) Patent Application Publication
(10) Pub. No.: US 2024/0011969 A1
(43) Pub. Date: Jan. 11, 2024

(54) Multi-Modal Multi-Spectral Imaging System and Method for Characterizing Tissue Types in Bladder Specimens

(71) Applicant: CytoVeris Inc., Farmington, CT (US)

(72) Inventors: Rishikesh Pandey, Unionville, CT (US); David Fournier, Northborough, MA (US); Patrick Curry, Glastonbury, CT (US); Alan Kersey, South Glastonbury, CT (US)

(21) Appl. No.: 18/271,023

(22) PCT Filed: Jan. 5, 2022
(86) PCT No.: PCT/US2022/011343

§ 371 Date: Jul. 5, 2023

Related U.S. Application Data
(60) Provisional application No. 63/134,145, filed on Jan. 5, 2021.

(51) Int. Cl. G01N 33/483; G01N 21/64

(52) U.S. Cl. CPC G01N 33/4833; G01N 21/6486; A61B 10/00

(57) ABSTRACT

A system and method for analyzing bladder tissue sample is provided that includes an excitation light source, a photodetector, an optical filter, and a system controller. The excitation light source produces excitation lights centered on a distinct wavelengths. At least one of the wavelengths produces autofluorescence emissions from one or more biomolecules, and at least one of the wavelengths produces diffuse reflectance signals. The photodetector detects at least one of the autofluorescence emissions or diffuse reflectance signals and produces signals representative thereof. The optical filter filters at least one of autofluorescence emissions or the diffuse reflectance signals. The system controller communicates with the system components and a memory storing instructions. The instructions cause the system controller to control the excitation light unit, receive and process the photodetector signals, produce a signal image, and analyze the tissue sample to determine the presence of detrusor muscle tissue.

This application claims priority to U.S. Patent Appln. No. 63/134,145 filed Jan. 5, 2021, which is hereby incorporated by reference in its entirety.

### BACKGROUND OF THE INVENTION

### 1. Technical Field

The present disclosure relates to devices and methods for identifying tissue samples in general, and the devices and methods for identifying bladder tissue samples in particular.

### 2. Background Information

Bladder cancer (“BC”) is the sixth most common cancer in the U.S. and approximately 75% of bladder tumors are classified as non-muscle-invasive bladder cancer (NMIBC). The standard treatment for NMIBC is complete transurethral resection of the bladder tumor (TURBT). The five-year recurrence rate for NMIBC after initial TURBT is 31-78%. [1] One reason that a repeat TURBT is often necessary is incomplete tumor resection. The American Urological Association/Society of Urologic Oncology (AUA/SUO) and the European Urologic Association (EUA) guidelines define complete resection as resection of the entire tumor extending to the bladder detrusor muscle (“DM”) wall. The depth of invasion can only be assessed accurately if all bladder wall layers, including the DM wall can be examined by a pathologist. The absence of muscle in the specimen is associated with a significantly higher risk of residual disease, early recurrence and tumor under-staging.

Accurate staging is an important prognostic factor for determining risk of recurrence and progression in BC. The current standard of care requires histopathological analysis of TURBT specimens. For adequate diagnosis, TURBT specimens must extend into the bladder muscle wall. For patients with high-grade BC, five-year cancer-specific mortality was 7.6%, 12.1% and 18.8%, respectively, when muscle was present, absent, or not mentioned. [2] For this reason, if there is not sufficient muscle in the specimen after the initial resection, guidelines recommend repeat TURBT. Almost half of TURBTs do not contain muscle as confirmed post-operatively by histopathologic examination. There are currently no practical tools available to surgeons to determine during the procedure whether the resected specimen includes sufficient muscle tissue. The availability of a rapid and accurate tool for point-of-care determination of detrusor muscle in TURBT will not only reduce cancer recurrence but will be prognostically significant. What is needed is a point-of-surgery imaging system for the in-vivo/ex-vivo examination of TURBT specimens.

A transurethral en bloc resection of a bladder tumor (“ERBT”) is a surgical approach that is an alternative to conventional TURBT. As compared to a TURBT approach that removes bladder tumor in a piece meal fashion, the ERBT approach is designed to remove the entirety of the tumor body as a single specimen. The whole ERBT specimen is preferable for pathological assessment of tumor depth and staging. What is needed is a point-of-surgery imaging system for the in-vivo/ex-vivo examination of ERBT specimens that will allow for an improved assessment of margin status for completeness of resection and depth of invasion.

### SUMMARY

According to an aspect of the present disclosure, a system for analyzing an ex-vivo bladder tissue sample is provided. The system includes an excitation light source, at least one photodetector, at least one optical filter, and a system controller. The excitation light source is configured to selectively produce a plurality of excitation lights. Each excitation light is centered on a wavelength distinct from the centered wavelength of the other excitation lights. At least one of the excitation light centered wavelengths is configured to produce autofluorescence emissions from one or more biomolecules associated with a bladder wall tissue, and at least one of the excitation light centered wavelengths is configured to produce diffuse reflectance signals from the tissue sample. The system is configured so that the plurality of excitation lights are incident to the tissue sample. The at least one photodetector is configured to detect the autofluorescence emissions, or the diffuse reflectance signals, or both from the tissue sample as a result of the respective incident excitation light, and to produce signals representative of the detected autofluorescence emissions, or the detected diffuse reflectance signals, or both. The at least one optical filter is operable to filter the signals representative of the detected autofluorescence emissions, or the detected diffuse reflectance signals, or both. The system controller is in communication with the excitation light unit, the at least one photodetector, and a non-transitory memory storing instructions, which instructions when executed cause the system controller to: a) control the excitation light unit to sequentially produce the plurality of excitation lights; b) receive and process the signals from the at least one photodetector for each sequential application of the plurality of excitation lights, and produce an image representative of the signals produced by each sequential application of the plurality of excitation lights; and c) analyze the tissue sample using a plurality of the images to determine the presence of detrusor muscle tissue within the tissue sample.

According to another aspect of the present disclosure, a system for analyzing an in-vivo bladder tissue sample is provided. The system includes an excitation light source, at least one photodetector, at least one optical filter, a probe, and a system controller. The excitation light source is configured to selectively produce a plurality of excitation lights. Each excitation light is centered on a wavelength distinct from the centered wavelength of the other excitation lights. At least one of the excitation light centered wavelengths is configured to produce autofluorescence emissions from one or more biomolecules associated with a bladder wall tissue, and at least one of the excitation light centered wavelengths is configured to produce diffuse reflectance signals from the tissue sample. The system is configured so that the plurality of excitation lights are incident to the tissue sample. The at least one photodetector is configured to detect the autofluorescence emissions, or the diffuse reflectance signals, or both from the tissue sample as a result of the respective incident excitation light, and to produce signals representative of the detected autofluorescence emissions, or the detected said diffuse reflectance signals, or both. The at least one optical filter is operable to filter the signals representative of the detected autofluorescence emissions, or the detected diffuse reflectance signals, or both. The probe is configured to be deployed within a bladder. The probe is in communication with the excitation light source and the at least one photodetector. The probe is configured to interrogate the bladder wall tissue with the plurality of excitation lights and to collect the autofluorescence emissions from one or more biomolecules associated with the bladder wall tissue and the diffuse reflectance signals from the bladder wall tissue. The system controller is in communication with the excitation light unit, the at least one photodetector, and a non-transitory memory storing instructions, which instructions when executed cause the system controller to: a) control the excitation light unit to sequentially produce the plurality of excitation lights; b) receive and process the signals from the at least one photodetector for each sequential application of the plurality of excitation lights, and produce an image representative of the signals produced by each sequential application of the plurality of excitation lights; and c) analyze the tissue sample using a plurality of the images to determine the presence of detrusor muscle tissue within the tissue sample.

According to another aspect of the present disclosure, a method of analyzing a bladder tissue sample is provided. The method includes: a) sequentially interrogating the tissue sample with a plurality of excitation lights, each excitation light centered on a wavelength distinct from the centered wavelength of the other excitation lights, wherein at least one of the excitation light centered wavelengths is configured to produce autofluorescence emissions from one or more biomolecules associated with a bladder wall tissue, and a diffuse reflectance signals from the tissue sample; b) using at least one photodetector to detect the autofluorescence emissions, or the diffuse reflectance signals, or both from the tissue sample, and to produce photodetector signals representative of the detected said autofluorescence emissions, or the detected said diffuse reflectance signals, or both; c) filtering the light emitted or reflected from the tissue sample resulting from each sequential interrogation of the tissue sample; d) processing the photodetector signals for each sequential application of the plurality of excitation lights, including producing an image representative of the photodetector signals produced by each sequential application of the plurality of excitation lights; and e) analyzing the tissue sample using each image to identify the presence of detrusor muscle tissue within the tissue sample.

According to an aspect of the present disclosure, a method of analyzing an in-vivo bladder wall tissue sample is provided. The method includes: a) inserting a probe into a subject's bladder; b) sequentially interrogating the bladder wall tissue sample with a plurality of excitation lights emanating from the probe, each excitation light centered on a wavelength distinct from the centered wavelength of the other excitation lights, wherein at least one of the excitation light centered wavelengths is configured to produce autofluorescence emissions from one or more biomolecules associated with a bladder wall tissue, and a diffuse reflectance signals from the tissue sample; c) using at least one photodetector to detect the autofluorescence emissions, or the diffuse reflectance signals, or both collected from the bladder wall tissue sample using the probe, and to produce photodetector signals representative of the detected autofluorescence emissions, or the detected said diffuse reflectance signals, or both; d) filtering the light emitted or reflected from the tissue sample resulting from each sequential interrogation of the tissue sample; e) processing the photodetector signals for each sequential application of the plurality of excitation lights, including producing an image representative of the photodetector signals produced by each sequential application of the plurality of excitation lights; and f) analyzing the bladder wall tissue sample using each image to identify the presence of detrusor muscle tissue within the bladder wall tissue sample.

In any of the aspects or embodiments described above and herein, the plurality of excitation lights may be in a UV wavelength range.

In any of the aspects or embodiments described above and herein, the plurality of excitation lights may be in the wavelength ranges of about 275-285 nm and about 345-371 nm and about 400-410 nm.

In any of the aspects or embodiments described above and herein, the excitation light source may include a plurality of excitation light units, and each excitation light unit may include at least one UV LED.

In any of the aspects or embodiments described above and herein, the autofluorescence emissions may be in the visible region.

In any of the aspects or embodiments described above and herein, the autofluorescence emissions may be in the wavelength ranges of about 352-388 nm, 380-420 nm, 429-475 nm, 532-552 nm and 593-643 nm.

In any of the aspects or embodiments described above and herein, the diffuse reflectance signals may be representative of light collected in the visible region.

In any of the aspects or embodiments described above and herein, the diffuse reflectance signals may be representative of light collected at about 405 nm, 450 nm, and 620 nm.

In any of the aspects or embodiments described above and herein, the instructions when executed may cause the system controller to analyze the tissue sample using each image to identify the presence of diseased tissue within the tissue sample, the analysis may include providing cellular or microstructural morphological information.

In any of the aspects or embodiments described above and herein, the tissue sample may be produced by a TURBT procedure, a ERBT procedure, a biopsy, or a cystectomy.

In any of the aspects or embodiments described above and herein, the system controller instructions may include at least one classifier that is trained using multispectral images.

In any of the aspects or embodiments described above and herein, at least a first of the multispectral images may be representative of the autofluorescence emissions and at least a second of the multispectral images may be representative of the diffuse reflectance signals.

In any of the aspects or embodiments described above and herein, the at least one of the biomolecules associated with the bladder wall tissue may include one or more of collagen, elastin. NADH, elastin, or hemoglobin.

In any of the aspects or embodiments described above and herein, wherein the probe may be configured to excise the bladder wall tissue sample.

In any of the aspects or embodiments described above and herein, the analyzing step may utilize one or more classifiers and at least one of the one or more classifiers may be trained using a library of bladder tissue samples that have been multispectrally analyzed with and without a histological stain.

In any of the aspects or embodiments described above and herein, the analyzing step may include empirically quantifying at least one of the biomolecules associated with a bladder wall tissue, the quantifying including determining a concentration of the at least one of the biomolecules associated with the bladder wall tissue.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagrammatic illustration of a present disclosure system embodiment.

FIG. 2 is a table of excitation/illumination wavelengths versus reflectance/fluorescence wavelengths.

FIG. 3 is a graph of fluorescence intensity versus fluorescence emission wavelength, illustrating diagrammatic representations of biomolecule curves.

FIG. 4 is a diagrammatic illustration of a bladder and includes an enlarged view of a bladder wall.

FIG. 5A is a flow diagram that schematically illustrates an example of a process for training a classifier that may be included in the present disclosure system.

FIG. 5B is a flow diagram that schematically illustrates an example of a process for analyzing a tissue sample using a classifier, for example that has been trained in the manner shown in FIG. 5A.

FIG. 6A is a flow diagram that schematically illustrates an example of a process for training a classifier that may be included in the present disclosure system.

FIG. 6B is a flow diagram that schematically illustrates an example of a process for analyzing a tissue sample using a classifier, for example that has been trained in the manner shown in FIG. 6A.

FIG. 7A is a flow diagram that schematically illustrates an example of a process for training a classifier that may be included in the present disclosure system.

FIG. 7B is a flow diagram that schematically illustrates an example of a process for analyzing a tissue sample using a classifier, for example that has been trained in the manner shown in FIG. 7A.

FIG. 8A is a flow diagram that schematically illustrates an example of a process for training a classifier that may be included in the present disclosure system.

FIG. 8B is a flow diagram that schematically illustrates an example of a process for analyzing a tissue sample using a classifier, for example that has been trained in the manner shown in FIG. 8A.

FIG. 9A illustrates an image of an excised tissue sample taken using a white light source.

FIG. 9B illustrates the excised tissue sample that is the subject of FIG. 10A after being subjected to a histological stain, imaged and the aforesaid image subjected to an affine transformation.

FIG. 9C illustrates the excised tissue specimen that is the subject of FIGS. 10A and 10B, as a multispectral mosaic produced by combining a plurality of multispectral images.

FIGS. 10A-10I illustrate an excised tissue sample imaged at a plurality of different excitation wavelengths to produce multispectral AF images and multispectral reflectance images.

FIG. 11 is a diagrammatic illustration of a present disclosure system embodiment that is configured for in-vivo applications.

### DETAILED DISCLOSURE

The present disclosure includes a novel dye-free multimodal optical approach that combines multispectral autofluorescence (“AF”) imaging with multispectral reflectance imaging to measure both tissue emission and absorption characteristics to provide comprehensive analysis and profiling of excised tissue; e.g., excised via TURBT or ERBT procedures. Unlike known multimodal devices which are fiber-based, the present disclosure allows wide-field optical imaging and can be used to provide near real-time acquisition and therefore can be used to provide diagnostic information during surgery. The present system utilizes multispectral AF imaging in at least two ways: a) imaging a sample in a series of wavelength bands for hyperspectral analysis; and b) using multiple excitation sources designed to preferentially and differentially excite certain biomolecular fluorophores.

The biomolecules present in different tissues provide discernible and repeatable AF [3-5] and reflectance [6] spectral patterns. The endogenous fluorescence signatures offer useful information that can be mapped to functional, metabolic, and/or morphological attributes of a biological specimen, and therefore may be used for diagnostic purposes. Biomolecular changes occurring in the cell and tissue state during pathological processes and disease progression result in alterations of the amount and distribution of endogenous fluorophores and can form the basis for tissue/cancer identification. Tissue AF has been proposed to detect various malignancies including cancer by measuring either differential intensity [7] or lifetimes of the intrinsic fluorophores [8]. Biomolecular constituents such as tryptophan, collagen, elastin, nicotinamide adenine dinucleotide (NADH), flavin adenine dinucleotide (FAD), elastin, porphyrins, etc. present in tissue provide discernible and repeatable autofluorescence spectral patterns.

AF spectroscopy has been proposed as a complementary tool to cystoscopy for the diagnosis of bladder cancer. [9] AF spectroscopy has been demonstrated to discern bladder cancer with sensitivity and specificity of 100%. [10] Moreover, a statistically significant difference in the redox ratio of healthy and bladder cancer cells indicative of a metabolic shift has been noted by Palmer et al. [11] While tissue AF has been proposed and demonstrated with varying degrees of success, there are several limitations for conventional AF-based diagnosis approaches. For example, traditional AF assays typically use a single excitation wavelength which will not excite all the intrinsic fluorophores present in the tissue. Consequently, a traditional AF assay does not effectively utilize the comprehensive and rich biochemical information embedded in the tissue matrix both from cells and extracellular matrix. As another example, most AF applications use a fiber probe with single point measurement capability and are inherently slow. [12,13] As another example, most AF approaches utilize relatively simple data analysis such as calculating redox ratio or oxygenation index ratio, and do not utilize the rich morphological information. As will be apparent from the description below, the present disclosure provides a novel, unobvious, and improved method and system that overcomes these limitations and others.

The present disclosure system includes an excitation light source, one or more optical filters, one or more photodetectors, and a system controller. In some embodiments, the system may include other components such as one or more of a filter controller, a scanning device, an optical switch, and the like. As will be described herein, embodiments of the present disclosure are configured for imaging/analysis of ex-vivo bladder tissue samples and other embodiments of the present disclosure may be configured for imaging/analysis of in-vivo bladder tissue. In those system embodiments configured for in-vivo analysis/imaging of bladder tissue, the system may include a modified resectoscope, or endoscope, or any other device that is used to visualize and excise tissues in a bladder that may be modified according to the present disclosure.

The excitation light source may include one or more excitation light units. In some embodiments, an excitation light unit may be configured to produce excitation light centered at a particular wavelength. In those system embodiments that include a plurality of excitation light units, different excitation light units may be configured to produce excitation light centered at different wavelengths; e.g., a first excitation light unit configured to produce excitation light centered at wavelength “X”, a second excitation light unit configured to produce excitation light centered at wavelength “Y”, and the like. In some embodiments, the excitation light source may be or include a white light source. For example, the system may include a white light source in combination with one or more filters that collectively produce excitation light centered at different wavelengths. In some embodiments, the system may include a white light source used to interrogate the sample unfiltered; e.g., for registration purposes, or the like.

An excitation light unit may be configured to produce AF emissions from a tissue sample and/or may be configured to produce reflectance signals from a tissue sample. Non-limiting examples of acceptable excitation light sources include lasers and light emitting diodes (LEDs) that may be centered at particular wavelengths, or a tunable excitation light source configured to selectively produce light centered at respective different wavelengths. An example of an acceptable white light source is a flash lamp. This disclosure is not limited to any particular type of excitation light unit. In those embodiments wherein an excitation light source is configured to produce light centered on a particular wavelength, the respective wavelength may be chosen based on the photometric properties associated with one or more biomolecules (or tissue type, etc.) of interest. Excitation light incident to a biomolecule that acts as a fluorophore will cause the fluorophore to emit fluorescent light at a wavelength longer than the wavelength of the excitation light; i.e., via AF.

As stated above, tissue may naturally include certain fluorophores such as tryptophan, collagen, elastin, nicotinamide adenine dinucleotide (NADH), flavin adenine dinucleotide (FAD), elastin, porphyrins, and the like. In addition, biomolecular changes occurring in the cell and tissue state during pathological processes and as a result of disease progression often result in alterations of the amount and distribution of these endogenous fluorophores. Hence, different tissue types and states can exhibit distinct intrinsic tissue AF, or in other words an “AF signature”, that is readily identifiable. Embodiments of the present disclosure may utilize these AF characteristics/signatures to identify different tissue types and/or tissue constituents.

Excitation wavelengths may also be chosen that cause detectable light reflectance from tissue of interest. The detectable light reflectance is a function of light absorption of the tissue and/or light scattering associated with the tissue (this may be collectively referred to as diffuse reflectance). Certain tissue types or permutations thereof, or constituents thereof, have differing and detectable light reflectance characteristics (“signatures”) at certain wavelengths. Significantly, these reflectance characteristics can provide information beyond intensity; e.g., information relating to cellular or microcellular structure such as cell nucleus and extracellular components. The morphology of a first type healthy tissue cell may be different from that of a second type healthy cell, and/or different from an abnormal or diseased tissue cell. Hence, the ability to gather cellular or microstructural morphological information (sometimes referred to as “texture”) provides another tool for determining tissue types and the state and characteristics of such tissue.

The excitation light source may be configured to produce light at wavelengths in the ultraviolet (UV) region (e.g., about 100-400 nm) and in some applications may include light in the visible region (e.g., 400-700 nm). The excitation light wavelengths may be chosen based on the absorption characteristics of the biomolecules of interest and the present disclosure is not, therefore, limited to the exemplary wavelength ranges disclosed above.

Regarding the one or more photodetectors within the system, the present disclosure may utilize a variety of different photodetector types configured to sense light and provide signals that may be used to measure the same. Non-limiting examples of an acceptable photodetector include those that convert light energy into an electrical signal such as photodiodes, avalanche photodiodes, a charge coupled device (“CCD”) array, an intensified charge coupled device (“ICCD”) array, a complementary metal-oxide-semiconductor (“CMOS”) image sensor, or the like. The photodetector may take the form of a camera. As will be described below, the photodetector(s) are configured to detect AF emissions from the interrogated tissue and/or diffuse reflectance from the interrogated tissue and produce signals representative of the detected light and communicate the signals to the system controller.

The system controller is in communication with system components such as the excitation light source and one or more photodetectors. In some system embodiments, the system may also be in communication with one or more of a: filter controller, a tunable optical filtering device, an optical switch, and the like as will be described below. The system controller may be in communication with these components to control and/or receive signals therefrom to perform the functions described herein. The system controller may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system to accomplish the same algorithmically and/or coordination of system components. The system controller includes or is in communication with one or more memory devices. The present disclosure is not limited to any particular type of memory device, and the memory device may store instructions and/or data in a non-transitory manner. Examples of memory devices that may be used include a computer readable storage medium, a read-only memory, random access memory, volatile memory, non-volatile memory, static memory, dynamic memory, flash memory, cache memory, and/or any device that stores digital information. The system controller may include, or may be in communication with, an input device that enables a user to enter data and/or instructions, and may include, or be in communication with, an output device configured, for example to display information (e.g., a visual display or a printer), or to transfer data, etc. Communications between the system controller and other system components may be via a hardwire connection or via a wireless connection.

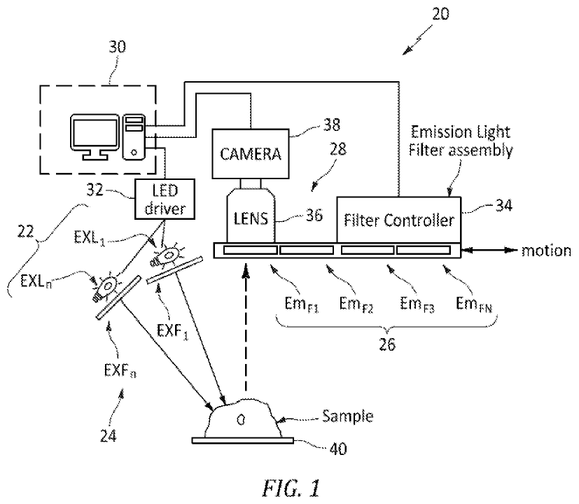

Embodiments of the present disclosure may include optical filtering elements configured to filter excitation light, or optical filtering elements configured to filter emitted light (including reflected light), or both. Each optical filtering element may be configured to pass a defined bandpass of wavelengths associated with an excitation light source or emitted/reflected light (e.g., fluorescence or reflectance), and may take the form of a bandpass filter. Regarding filtering excitation light, the system may include an independent filtering element associated with each independent excitation light source or may include a plurality of filtering elements disposed in a movable form (e.g., a wheel or a linear array configuration) or may include a single filtering element that is operable to filter excitation light at a plurality of different wavelengths, or each excitation light source may be configured to include a filtering element, or the like. Regarding filtering emitted or reflected light, the system may include a plurality of independent filtering elements each associated with a different bandwidth or may include a plurality of filtering elements disposed in a movable form or may include a single filtering element that is operable to filter emitted/reflected light at a plurality of different wavelengths, or the like. The bandwidth of the emitted/reflected light filters are typically chosen based on the photometric properties associated with one or more biomolecules of interest. Certain biomolecules may have multiple emission or reflectance peaks. The bandwidth of the emitted/reflected light filters are typically chosen to allow only emitted/reflected light from a limited portion of the biomolecule emission/reflectance response; i.e., a portion of interest that facilitates the analysis described herein. As will be described below, the exemplary system embodiment shown in FIG. 1 illustrates a non-limiting example of optical filtering. In some embodiments, the system may include a tunable bandpass filter that is controllable to provide a plurality of different bandwidth filtration modes. In certain embodiments, the excitation filter may be disposed or integrated as a part of excitation light source. For example, the LED or other light source can be coated with a material to allow desired bandpass.

An exemplary embodiment of a present disclosure system 20 is diagrammatically illustrated in FIG. 1. This system 20 embodiment includes an excitation light source 22, an excitation light filter arrangement 24, an emission/reflectance light filter assembly 26, a photodetector arrangement 28, and a system controller 30. The excitation light source 22 includes a plurality of independent excitation light sources (e.g., EXL₁ . . . EXLₙ, where “n” is an integer greater than one), each operable to produce an excitation light centered at a particular wavelength and each centered on an excitation wavelength different from the others. The independent excitation light sources are directly or indirectly in communication with the system controller 30. In this example, the independent excitation light sources are UV LEDs. As described above, the wavelengths produced by the independent excitation light sources are chosen based on the photometric properties associated with biomolecules/tissue types of interest. The LEDs are in communication with an LED driver 32 that may be independent of the system controller 30 or the functionality of the LED driver 32 may be incorporated into the system controller 30. The excitation light filter arrangement 24 shown in FIG. 1 includes an independent bandpass filter (EXF₁ . . . EXFₙ) for each excitation light source and the bandwidth filter properties for each independent bandpass filter are tailored for the respective excitation light source with which it is associated. In alternative embodiments the system 20 may be configured without an excitation light filter arrangement 24, or each excitation light source may have an incorporated filter element, or the system 20 may include an excitation light filter arrangement 24 with a movable filter element (e.g., a wheel, linear array, etc.), or may include a single filtering element that is operable to filter excitation light at a plurality of different wavelengths. The system 20 embodiment diagrammatically shown in FIG. 1 includes an emission light filter assembly 26 having a filter controller 34 and a linear array of bandpass filters (e.g., Em_{F1}, Em_{F2} . . . Em_{FN}). The filter controller 34 is configured to selectively position each respective bandpass filter in a light path between the tissue sample (i.e., the source of the emitted/reflected light) and the photodetector arrangement 28 to permit filtering of the emitted/reflected light prior to detection by the photodetector arrangement 28. The filter controller 34 may be in communication with the system controller 30, or the filter controller 34 functionality may be incorporated into the system controller 30. As stated above, the bandwidth of the respective bandpass filters for the emitted/reflected light are typically chosen based on the photometric properties associated with one or more biomolecules of interest; e.g., to allow only emitted/reflected light from a limited portion of the biomolecule emission/reflectance response that is of interest to facilitate the analyses described herein. The photodetector arrangement 28 includes a lens arrangement 36 and a camera 38. The lens arrangement 36 is configurable to suit the application at hand. For example, in some embodiments the lens arrangement 36 may include a single fixed focus lens. In some embodiments, the lens arrangement 36 may be configured to address chromatic dispersion. For example, the lens arrangement 36 may include one or more corrective lenses configured to address aberration/focus as may be desired. In some embodiments, the lens arrangement 36 may be controllable to selectively change lens configurations and is in communication with the system controller 30. The camera 38 is configured to produce signals representative of the sensed emitted/reflected light passed through the emission light filter assembly 26. The aforesaid signals may be referred to as an “image” or may be processed into an image. The camera 38 is in communication with the system controller 30.

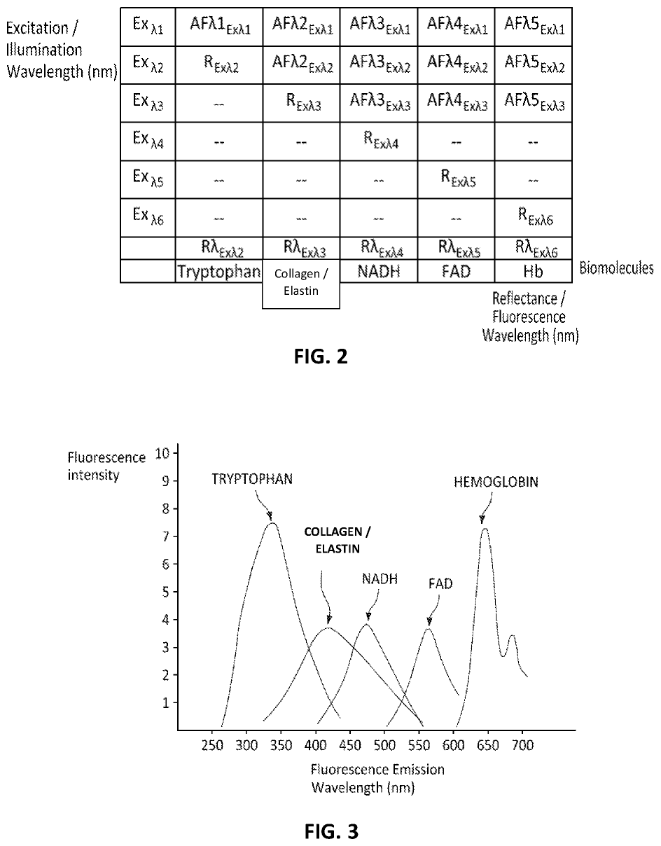

In the operation of the system 20 embodiment diagrammatically shown in FIG. 1, an excised tissue sample may be placed on a stage 40 or other platform at a position optically aligned with the photodetector arrangement 28. In some instances, the system 20 and/or the tissue sample may be such that the entirety of the sample can be imaged without changing the relative positions of the tissue sample and the system optics. In other instances, wherein the system 20 is not configured to image the entirety of the tissue sample, the system 20 may be configured to move one or both of the tissue sample and the system optics relative to one another so multiple regions of the tissue sample may be imaged; e.g., the tissue sample may be scanned. The images from the respective regions may subsequently be “stitched” together to form one or more images of the entirety of the tissue sample. In some instances, the stage 40 may include a plurality of fiduciary markers to facilitate registration between images. The system controller 30 (through stored instructions) is configured to sequentially operate the independent excitation light sources (e.g., EXL₁ . . . EXLₙ). As each excitation light source is operated, the produced excitation light passes through an excitation light filter prior to being incident to the tissue sample. If a fluorophore of interest is present within the tissue sample and that fluorophore is responsive to the wavelength of the incident excitation light, the excitation light will cause the fluorophore to produce an AF emission at a wavelength that is different from the excitation wavelength. Excitation light centered on a particular wavelength may produce AF emissions from more than one fluorophore of interest. Referring to the table in FIG. 2, a first excitation wavelength (EXλ1) may produce AF emissions at several different wavelengths (AFλ1_{EXλ1}, AFλ2_{EXλ1}, AFλ3_{EXλ1}, AFλ4_{EXλ1}, AFλ5_{EXλ1}). The same excitation light incident to the tissue sample may also generate diffuse reflectance signals; i.e., excitation light that is reflected from the tissue sample. For example, and again referring to the table in FIG. 2, a second excitation wavelength (EXλ2) can produce reflectance signals (R_{EXλ2}) and AF emissions at several different wavelengths (AFλ2_{EXλ2}, AFλ3_{EXλ2}, AFλ4_{EXλ2}, AFλ5_{EXλ2}), a third excitation wavelength (EXλ3) can produce reflectance signal (R_{EXλ3}) and AF emissions at several different wavelengths (AFλ3_{EXλ3}, AFλ4_{EXλ3}, AFλ5_{EXλ4}), and so on. The emission/reflectance light filter assembly 26 is controlled to coordinate placement of a particular bandpass filter in alignment with the camera 38, which bandpass filter is appropriate for the excitation light source being operated and to produce a limited bandwidth of the emitted/reflected light that is of interest for the analysis at hand; e.g., associated with particular biomolecules of interest. Some amount of the emitted light passes through the bandpass filter, is sensed by the camera 38, and the camera 38 produces signals representative of the sensed emitted/reflected light. The aforesaid signals may be referred to as an image or may be processed into an image. In some applications, an excitation wavelength may be chosen only for AF emissions of interest (e.g., EXλ1 in FIG. 2), and/or an excitation wavelength may be chosen only for diffuse reflectance signals of interest (e.g., EXλ4, EXλ5, and EXλ6 in FIG. 2). The above described process is repeated until the sample has been examined using all of the desired wavelengths of excitation light. As will be detailed below, the respective images may be used to collectively identify biomolecules/tissue types of interest with a desirable degree of specificity and sensitivity.

In the system embodiment described above and others, the signals (i.e., image) representative of the emitted light (AF and/or reflectance) captured by the photodetector arrangement (e.g., camera or plurality of photodetectors) for each excitation light wavelength may collectively provide a mosaic of information relating to the tissue sample. The chart shown in FIG. 2 illustrates an exemplary scenario wherein five (5) different excitation light sources, each centered on a different wavelength (i.e., Ex_{λ1}, Ex_{λ2}, Ex_{λ3}, Ex_{λ4}, Ex_{λ5}, and Ex_{λ6} nm), are used within the system. Depending on the presence of certain fluorophores within the tissue sample, the first excitation wavelength (i.e., Ex_{λ1}) may produce AF emissions of interest at five (5) different wavelengths (AFλ1_{Exλ1}, AFλ2_{Exλ1}, AFλ3_{Exλ1}, AFλ4_{Exλ1}, AFλ5_{Exλ1}), and the second excitation wavelength (i.e., Ex_{λ2}) may produce AF emissions of interest at four (4) different wavelengths (AFλ2_{Exλ2}, AFλ3_{Exλ2}, AFλ4_{Exλ2}, AFλ5_{Exλ2}), and so on. The second excitation wavelength (i.e., Ex_{λ2}) may also produce a reflectance image at this wavelength (R_{Exλ2}) that is a useful indicator of the presence or absence of certain tissue types within the tissue sample. The E_{xλ4}, Ex_{λ5}, and Ex_{λ6} excitation wavelengths may not be used to produce AF emissions of interest, but each may be used to produce a reflectance image of interest (i.e., R_{Exλ4}, R_{Exλ5}, R_{Exλ6}). As can be seen from the example shown in FIG. 2, the six (6) excitation wavelengths (i.e., Ex_{λ1}, Ex_{λ2}, Ex_{λ3}, Ex_{λ5}, and Ex_{λ6} nm) may be used to produce seventeen emitted light images (AFλ1_{Exλ1}, AFλ2_{Exλ1}, AFλ3_{Exλ1}, AFλ4_{Exλ1}, AFλ5_{Exλ1}, R_{Eλ2}, AFλ2_{Exλ2}, AFλ3_{Exλ2}, AFλ4_{Exλ2}, AFλ5_{Exλ2}, R_{Exλ3}, AFλ3_{Exλ3}, AFλ4_{Exλ3}, AFλ5_{Eλ3}, R_{Exλ4}, R_{Exλ5}, R_{Exλ6}) that may be used collectively to identify biomolecule/tissue types of interest with a desirable degree of specificity and sensitivity. It should be noted that the number of excitation wavelengths, the number of reflectance wavelengths, the biomolecule, and the particular AF emissions selected, and reflectance emissions indicated in FIG. 2 are provided to illustrate the present disclosure, and the present disclosure is not limited to this example. For example, the analysis of different types of tissue may benefit from fewer or more excitation wavelengths, different biomolecule, etc.

The integrated information provided by the aforesaid emitted light images provide distinct benefits in the process of identifying biomolecule/tissue types of interest with a desirable degree of specificity and sensitivity. As can be seen from FIG. 3, AF emissions are produced in a peaked band with an intensity value that is centered on a particular wavelength. Hence, AF emissions centered on a particular wavelength will include AF emissions not only on the peak wavelength but also on adjacent wavelengths albeit at a lesser intensity. As can also be seen in FIG. 3, the biomolecule/fluorophores of interest (e.g., tryptophan, collagen, NADH, FAD, elastin, hemoglobin, etc.) have characteristic AF intensity curves with a peak centered on a wavelength but also including lesser intensities at wavelengths adjacent the peak wavelength. The AF intensity curves of some of the biomolecules may overlap to a degree. As a result, AF emissions at a particular wavelength within the overlap region may be a product of AF emissions from a first biomolecule or from a second biomolecule and are likely not dispositive by themselves of either biomolecule. As indicated above, at least some biomolecule of interest also have reflectance curves (indicating the amount of light reflectance which is a function of light absorption of the tissue and light scattering within the tissue) with a peak centered on a peak wavelength but also including lesser intensities at wavelengths adjacent the peak wavelength. The reflectance curves of some of the biomolecules may also overlap to a degree. As a result, reflectance at a particular wavelength within the overlap region may be a product of reflectance from a first biomolecule or from a second biomolecule and is likely not dispositive by itself of either biomolecule. In addition, as indicated above, reflectance images can also provide cellular or tissue microstructrual information that can be used as an additional tool for determining tissue types and the state of such tissue. The collective information provided by the aforesaid plurality of emitted/reflected light images produced by the present disclosure system, however, provides distinct information at different excitation wavelengths that can be used to identify biomolecule/tissue types with a desirable degree of specificity and sensitivity.

In some embodiments, the stored instructions within the system controller 30 may include an artificial intelligence/machine learning (AI/ML) algorithm trained classifier that is “trained” using a clinically significant number of images of known tissue types (e.g., bladder wall tissue types and components including muscularis propria, pervesical fat, lamina propria, urothelium, diseased tissue, adipose, etc.) and features collected at the respective excitation wavelengths. Alternatively, the system controller 30 may be in communication with such an artificial intelligence/machine learning (AI/ML) algorithm trained classifier. The trained classifier in turn may be used to evaluate the acquired light images (AF and/or reflectance) collected from the tissue sample at the various different excitation/emission wavelengths to determine the presence or absence of biomolecule/tissue types/features of interest. A dictionary learning, anomaly detector, convolutional neural network (CNN), deep neural network (DNN), or a random forest type classifier are examples algorithms that may be used. In some embodiments, an ensemble classifier consisting of two or more classifiers based on the same or different classification methods can be utilized. The present disclosure is not limited to these examples.

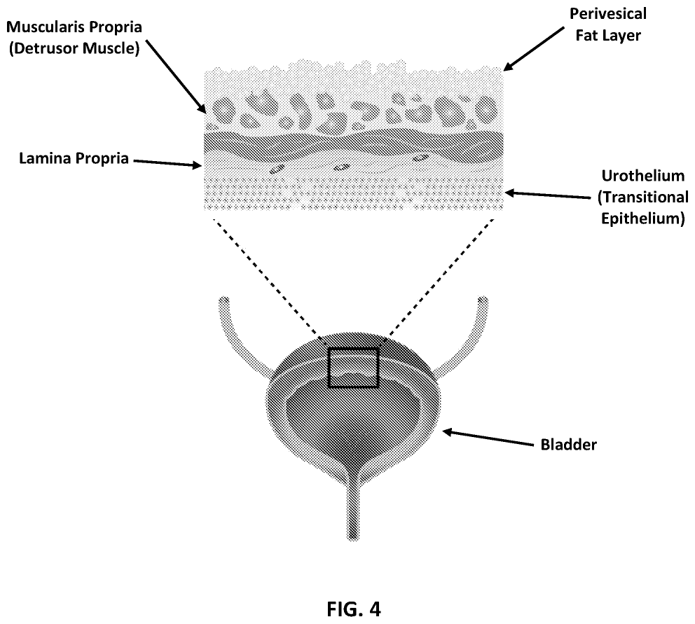

The present disclosure permits the identification of bladder tissue types and thereby provides significant utility, for example, in evaluating a tissue sample resected from a bladder. As indicated above, the depth of invasion (e.g., of diseased tissue) can only be assessed accurately if all bladder wall layers, including the DM wall, can be examined. For at least that reason, current guidelines define a complete resection as a resection of the entirety of a tumor extending to the bladder detrusor muscle (“DM”) wall. FIG. 4 diagrammatically illustrates a bladder and includes an enlarged view of a bladder wall with the respective wall layers labeled to facilitate the discussion herein. The present disclosure includes a novel dye-free multimodal optical approach that combines multispectral AF imaging with multispectral reflectance imaging (as described above) to measure both tissue emission and absorption characteristics to provide comprehensive analysis and profiling of bladder tissue excised via TURBT or ERBT procedures, or by biopsy, or by cystectomy, or the like. In addition to ex-vivo applications, embodiments of the present disclosure may also be configured for in-vivo applications as described herein.

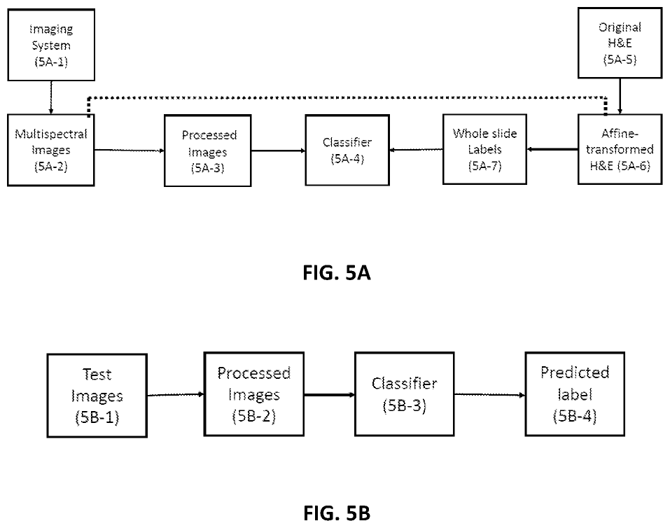

Embodiments of the present disclosure utilize artificial intelligence to facilitate an accurate, automated, and rapid analysis of an excised tissue sample. As indicated above, embodiments of the present disclosure system may include a system controller having stored instructions that include an artificial intelligence/machine learning (AI/ML) algorithm trained classifier, or alternatively the system controller 30 may be in communication with such a classifier. The flow diagram of FIG. 5A schematically illustrates an example of a process for training a classifier that may be included in the present disclosure system.

During the training process, an imaging system (e.g., the same as or similar to the photodetectors described above in FIG. 1—e.g., a camera) is used to acquire a clinically significant number of multispectral images (see boxes 5A-1 and 5A-2). The “clinically significant number of images” may vary depending on the classifier(s) used and the target tissue type and/or target tissue constituent but will include a number of images adequate to enable the classifier to perform with an acceptable degree of certainty for the task at hand. As stated above, embodiments of the present disclosure may use multispectral AF imaging and multispectral reflectance imaging to measure both tissue absorption and emission/reflectance characteristics. The multispectral AF imaging may include sequentially interrogating the tissue sample with light centered on a “N” number of different wavelengths, where “N” is an integer and is equal to or greater than one. In similar fashion, the multispectral reflectance imaging may include sequentially interrogating the tissue sample with light centered on a “M” number of different wavelengths, where “M” is an integer and is equal to or greater than one. To be clear and as described above, in some instances a tissue type may be identified using only multispectral AF, or only multispectral reflectance, or tissue identification may be accomplished using some combination of multispectral AF and multispectral reflectance. The acquired images may be processed to provide desirable uniformity to the respective images (box 5A-3). The processing may include, but is not limited to, altering the images to a uniform size or format, adjusting image intensity to a uniform level, aligning the images, and the like. The processed images may then be input to the classifier (box 5A-4). Subsequent to the tissue sample being imaged (as described in boxes 5A-1 and 5A-2), the same tissue sample may be subjected to a histological stain (box 5A-5). A non-limiting example of such a histological stain is hematoxylin and eosin stain (commonly referred to as “H&E” stain). The stained tissue sample may then be imaged and the images of the stained tissue sample may be transformed (box 5A-6) to facilitate a comparison between the H&E stain images and the acquired multispectral images. An example of a transformation that may be used is an affine transformation that preserves geometric relationships of aspects of the H&E images. The transformed images may then be read by a pathologist (box 5A-7). The pathological analysis of each image may include a tissue type determination based on the entirety of the respective image or may include a read of segments of respective images, or features present within the respective images, or the like. Non-limiting examples of features include intensity, intensity ratios, texture information, and the like. The transformed H&E stained images and the respective pathological analysis of each image may then be input into the classifier (box 5A-4) for comparison with the processed images (box 5A-3).

The flow diagram of FIG. 5B schematically illustrates an example of a process for analyzing a tissue sample using a trained classifier; e.g., a classifier trained as described above regarding FIG. 5A. An imaging system (e.g., the same as or similar to the photodetectors described above in FIG. 1—a camera) is used to acquire a number of multispectral images (box 5B-1) from a tissue sample. As stated above, embodiments of the present disclosure may use multispectral AF imaging and multispectral reflectance imaging to measure both tissue absorption and emission characteristics. The acquired images may be processed to provide desirable uniformity to the respective images (box 5B-2) as described above with respect to FIG. 5A. The processed images may then be input to the classifier portion of the system (box 5B-3). The system may then provide a determination or information regarding the type of tissue present in the imaged tissue sample (box 5B-4).

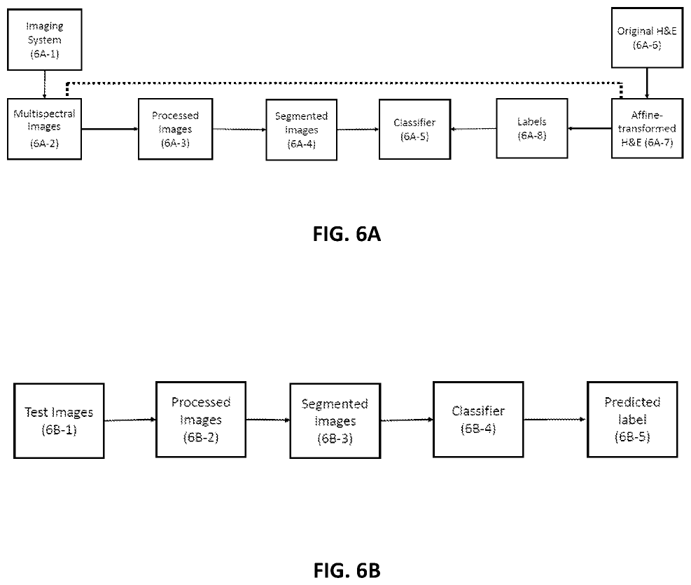

The flow diagram of FIG. 6A schematically illustrates a modified example of the process for training a classifier shown in FIG. 5A that may be included in the present disclosure system. In this example, aspects of the training process are similar to that described above with respect to FIG. 5A. For example, an imaging system is used to acquire a clinically significant number of multispectral images (see boxes 6A-1 and 6A-2). Here again, a tissue type may be identified using only multispectral AF, or only multispectral reflectance, or tissue identification may be accomplished using some combination of multispectral AF and multispectral reflectance. The acquired images may be processed to provide desirable uniformity to the respective images (box 6A-3). In this exemplary training process, the processed images may be evaluated to identify one or more segments (e.g., portions of the images) that contain a region of interest that may be used to facilitate tissue identification (box 6A-4). The segmented images (and/or the image segments) may then be input to the classifier (box 6A-5). Again, similar to the process illustrated in FIG. 5A, subsequent to the tissue sample being imaged (as described in boxes 6A-1 and 6A-2), the same tissue sample may be subjected to a histological stain (box 6A-6). The stained tissue sample may then be imaged and the images of the stained tissue sample may be transformed (box 6A-7) to facilitate a comparison between the H&E stain images and the acquired multispectral images. The transformed images (and/or the segmented image portions) may then be read by a pathologist (box 6A-8). The pathological analysis of each image segment may provide information that may be used for tissue identification purposes. The transformed H&E stained images and the respective pathological analysis of each segmented image may then be input into the classifier (box 6A-5) for comparison with the segmented images (box 6A-4).

The flow diagram of FIG. 6B schematically illustrates an example of a process for analyzing a tissue sample using a trained classifier; e.g., a classifier trained as described above regarding FIG. 6A. An imaging system is used to acquire a number of multispectral images (box 6B-1) from a tissue sample. As stated above, embodiments of the present disclosure may use multispectral AF imaging and/or multispectral reflectance imaging. The images may be processed as described above (box 6B-2). The processed images may be evaluated to identify one or more segments (e.g., portions of the images) that contain a region of interest that may be used to facilitate tissue identification (box 6B-3). The processed segmented images may then be input to the classifier portion of the system (box 6B-4). The system may then provide a determination or information regarding the type of tissue present in the imaged tissue sample (box 6B-5).

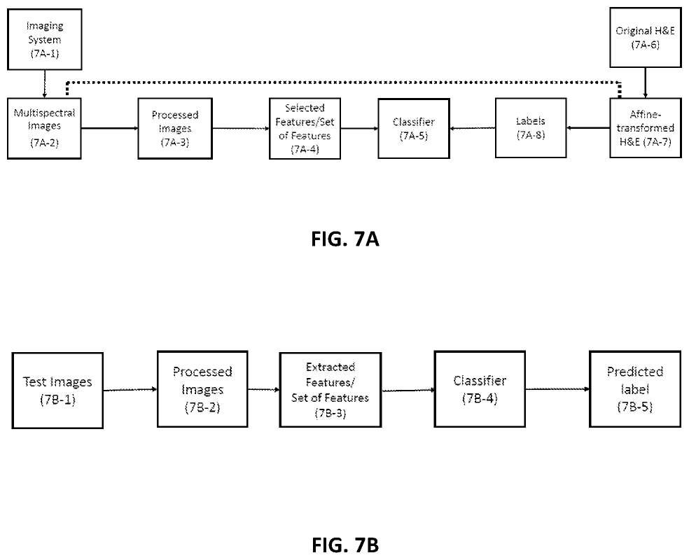

The flow diagram of FIG. 7A schematically illustrates another modified example of the process for training a classifier shown in FIGS. 5A and 6A that may be included in the present disclosure system. In this example, aspects of the training process are similar to that described above with respect to FIGS. 5A and 6A. For example, an imaging system is used to acquire a clinically significant number of multispectral images (see boxes 7A-1 and 7A-2). Here again, a tissue type may be identified using only multispectral AF, or only multispectral reflectance, or tissue identification may be accomplished using some combination of multispectral AF and multispectral reflectance. The acquired images may be processed to provide desirable uniformity to the respective images (box 7A-3). In this exemplary training process, the processed images may be evaluated to identify one or more tissue sample features that may be used to facilitate tissue identification (box 7A-4). The aforesaid features may be extracted from the respective images or otherwise made identifiable within the respective images. The processed images (and/or the image features) may then be input to the classifier (box 7A-5). Again, similar to the process illustrated in FIGS. 5A and 6A, subsequent to the tissue sample being imaged (as described in boxes 7A-1 and 7A-2), the same tissue sample may be subjected to a histological stain (box 7A-6). The stained tissue sample (or just certain features) may then be imaged and the images of the stained tissue sample (and/or features) may be transformed (box 7A-7) to facilitate a comparison between the H&E stain images and the acquired multispectral images. The transformed images (and/or the image features) may then be read by a pathologist (box 7A-8). The pathological analysis of each image feature may provide information that may be used for tissue identification purposes. The transformed H&E stained images and the respective pathological analysis of each image feature may then be input into the classifier (box 7A-5) for comparison with the images of the extracted features (box 7A-4).

The flow diagram of FIG. 7B schematically illustrates an example of a process for analyzing a tissue sample using a trained classifier; e.g., a classifier trained as described above regarding FIG. 7A. An imaging system is used to acquire a number of multispectral images (box 7B-1) from a tissue sample. As stated above, embodiments of the present disclosure may use multispectral AF imaging and/or multispectral reflectance imaging. The images may be processed as described above (box 7B-2). The processed images may be evaluated to identify one or more features that may be used to facilitate tissue identification (box 7B-3). The processed images containing the features (and/or the extracted features) may then be input to the classifier portion of the system (box 7B-4). The system may then provide a determination or information regarding the type of tissue present in the imaged tissue sample (box 7B-5).

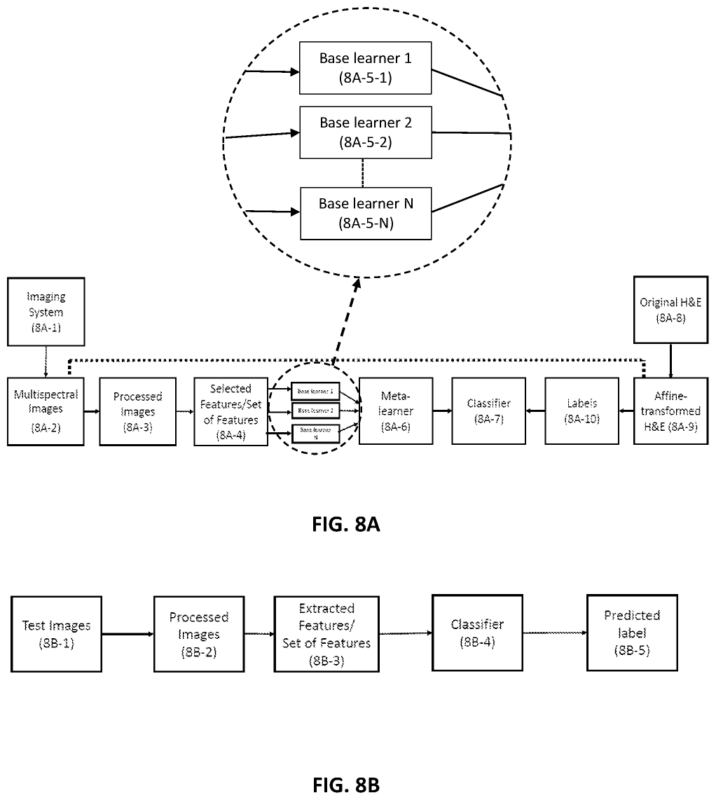

The flow diagram of FIG. 8A schematically illustrates another modified example of the process for training a classifier shown in FIGS. 5A, 6A, and 7A that may be included in the present disclosure system. In this example, aspects of the training process are similar to that described above with respect to FIGS. 5A, 6A, and 7A. For example, an imaging system is used to acquire a clinically significant number of multispectral images (see boxes 8A-1 and 8A-2). Here again, a tissue type may be identified using only multispectral AF, or only multispectral reflectance, or tissue identification may be accomplished using some combination of multispectral AF and multispectral reflectance. The acquired images may be processed to provide desirable uniformity to the respective images (box 8A-3). In this exemplary training process, the processed images may be evaluated to identify one or more tissue sample features that may be used to facilitate tissue identification (box 8A-4). The aforesaid features may be extracted from the respective images or otherwise made identifiable within the respective images. Respective features (e.g., 1 through “N” number of features) may then each be subjected to an additional classifier (the same or a different type of classifier), respectively labeled “Base learner 1, Base learner 2 . . . Base learner N” (see boxes 8A-5-1, 8A-5-1 . . . 8A-5-N). The images (and/or the features) classified in the base learners may then be input to a meta learner (box 8A-6) for further classification. The images (and/or the features) classified in the meta learner may then be input to a subsequent classifier (8A-7). Again, similar to the process illustrated in FIGS. 5A, 6A, and 7A, subsequent to the tissue sample being imaged (as described in boxes 8A-1 and 8A-2), the same tissue sample may be subjected to a histological stain (box 8A-8). The stained tissue sample (or just certain features) may then be imaged and the images of the stained tissue sample (and/or features) may be transformed (box 8A-9) to facilitate a comparison between the H&E stain images and the acquired multispectral images. The transformed images (and/or the image features) may then be read by a pathologist (box 8A-10). The pathological analysis of each image feature may provide information that may be used for tissue identification purposes. The transformed H&E stained images and the respective pathological analysis of each image feature may then be input into the classifier (box 8A-7) for comparison with the images of the extracted features (box 8A-6).

The flow diagram of FIG. 8B schematically illustrates an example of a process for analyzing a tissue sample using a trained classifier; e.g., a classifier trained as described above regarding FIG. 8A. An imaging system is used to acquire a number of multispectral images (box 8B-1) from a tissue sample. As stated above, embodiments of the present disclosure may use multispectral AF imaging and/or multispectral reflectance imaging. The images may be processed as described above (box 8B-2). The processed images may be evaluated to identify one or more features that may be used to facilitate tissue identification (box 8B-3). The processed images containing the features (and/or the extracted features) may then be input to the classifier portion of the system (box 8B-4). The system may then provide a determination or information regarding the type of tissue present in the imaged tissue sample (box 8B-5).

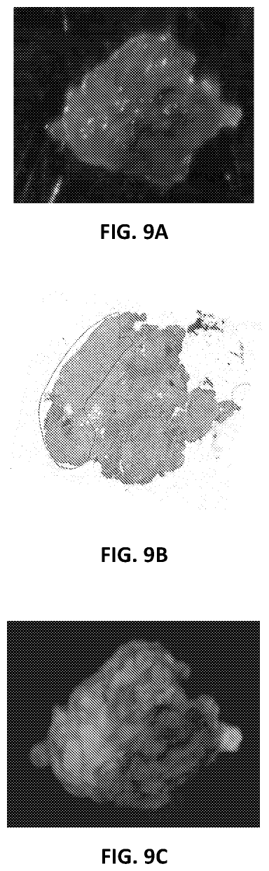

FIG. 9A illustrates an image of an excised tissue sample taken using a white light source. FIG. 9B illustrates the same excised tissue sample after being subjected to a histological stain, imaged and the aforesaid image subjected to an affine transformation. FIG. 9C illustrates a multispectral mosaic produced by combining a plurality of AF images (e.g., at one or more different excitation wavelengths) and reflectance images (e.g., at one or more different excitation wavelengths). The information available via the present disclosure from the multispectral mosaic image, the H&E image and the white light image is comprehensive and rich biochemical information that greatly enhances an automated, accurate, and rapid identification of tissue types.

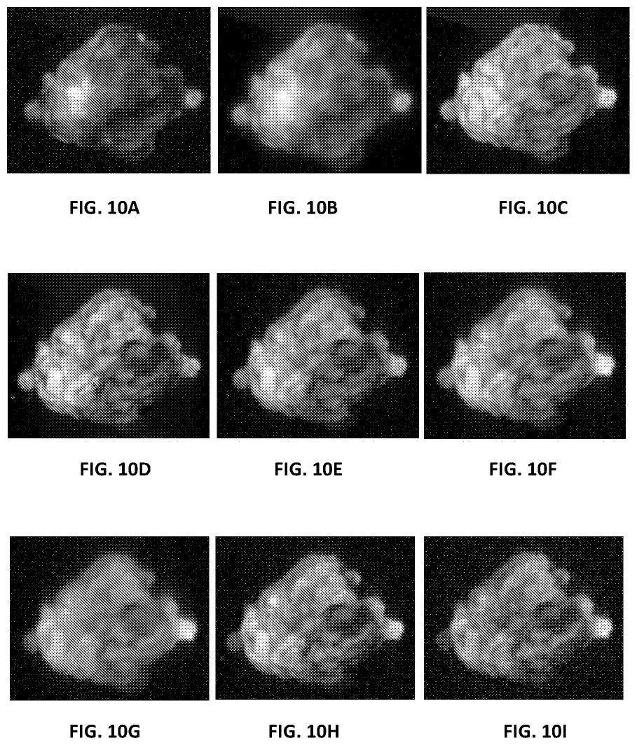

FIGS. 10A-10I illustrate an excised tissue sample imaged at a plurality of different excitation wavelengths to produce multispectral AF images and multispectral reflectance images that may be used according to the present disclosure as described herein. The information available from these multispectral image (individually or collectively; e.g., as mosaics) is comprehensive and greatly facilitates an automated, accurate, and rapid identification of tissue types.

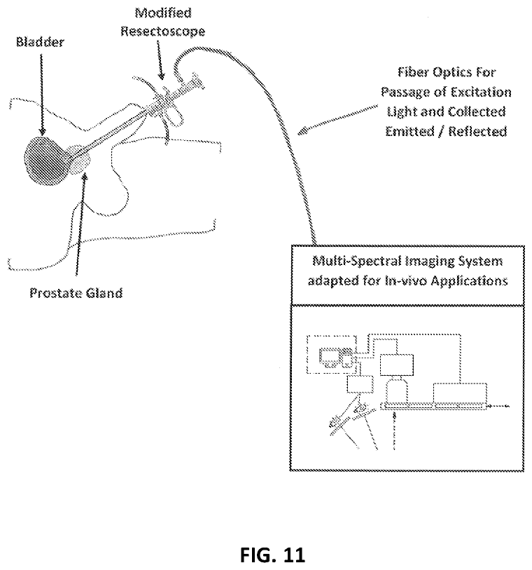

Referring to FIG. 11, as indicated above, aspects of the present disclosure may be configured for use in examining bladder tissue in an in-vivo application. In these system embodiments the system may include a device (e.g., a “probe”) that is used to visualize and excise tissues in a bladder including the entirety of the bladder. Non-limiting examples of such a device include a resectoscope, an endoscope, a biopsy needle, or the like that may be modified to permit the communication of excitation light to the bladder tissue of interest (e.g., the bladder wall tissue unresected, or the bladder wall tissue post-resection, etc.) and collection of emitted and/or reflected light resulting from the present system's multispectral light interrogation as described herein. In cystectomy applications, the present system may include a probe configured to permit the communication of excitation light to and collection of light from the tissue of interest (e.g., tissue formerly contiguous with the removed bladder) via fiber optics or other light conduit for analysis purposes to ensure satisfactory margins are present. FIG. 11 diagrammatically illustrates an embodiment of the present disclosure configured for an in-vivo application that includes a modified resectoscope. This example is provided for illustration purposes and the present disclosure is not limited thereto.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements.

### REFERENCES

1. A. G. van der Heijden et al., “Recurrence, Progression, and Follow-Up in Non—Muscle-Invasive Bladder Cancer”, European Urology Supplements 8, 556-562 (2009).
2. S. S. Chang et al., “Diagnosis and Treatment of Non-Muscle Invasive Bladder Cancer: AUA/SUO Guideline”, J Urol 196, 1021-1029 (2016).
3. R. A. Schwarz et al., “Autofluorescence and diffuse reflectance spectroscopy of oral epithelial tissue using a depth-sensitive fiber-optic probe”, Appl Opt 47, 825-834 (2008).
4. W. Zheng et al., “Optimal excitation-emission wavelengths for autofluorescence diagnosis of bladder tumors”, Int J Cancer 104, 477-481 (2003).
5. T. A. Valdez et al., “Multiwavelength fluorescence otoscope for video-rate chemical imaging of middle ear pathology”, Anal Chem 86, 10454-10460 (2014).
6. T. M. Bydlon et al., “Chromophore based analyses of steady-state diffuse reflectance spectroscopy: current status and perspectives for clinical adoption”, J Biophotonics 8, 9-24 (2015).
7. M. Wang et al., Autofluorescence Imaging and Spectroscopy of Human Lung Cancer. Applied Sciences 7, 32 (2017).
8. M. Marsden et al., “Intraoperative Margin Assessment in Oral and Oropharyngeal Cancer Using Label-Free Fluorescence Lifetime Imaging and Machine Learning”, IEEE Transactions on Biomedical Engineering 68, 857-868 (2021).
9. K. Bochenek et al., “Methods for bladder cancer diagnosis—The role of autofluorescence and photodynamic diagnosis”, Photodiagnosis Photodyn Ther 27, 141-148 (2019).
10. W. Zheng et al, “Optimal excitation-emission wavelengths for autofluorescence diagnosis of bladder tumors”, Int J Cancer 104, 477-481 (2003).
11. S. Palmer at al., (2015) “Discrimination of healthy and cancer cells of the bladder by metabolic state, based on autofluorescence”, in Proc. SPIE.
12. R. A. Schwarz et al., “Autofluorescence and diffuse reflectance spectroscopy of oral epithelial tissue using a depth-sensitive fiber-optic probe”, Appl. Opt. 47, 825-834 (2008).
13. Z. Volynskaya et al., “Diagnosing breast cancer using diffuse reflectance spectroscopy and intrinsic fluorescence spectroscopy”, J Biomed Opt 13, 024012 (2008).

1. A system for analyzing an ex-vivo bladder tissue sample, comprising:
an excitation light source configured to selectively produce a plurality of excitation lights, each said excitation light centered on a wavelength distinct from the centered wavelength of the other said excitation lights, wherein at least one of the excitation light centered wavelengths is configured to produce autofluorescence emissions from one or more biomolecules associated with a bladder wall tissue, and a diffuse reflectance signals from the tissue sample, the system configured so that the plurality of excitation lights are incident to the tissue sample;
at least one photodetector configured to detect the autofluorescence emissions, or the diffuse reflectance signals, or both from the tissue sample as a result of the respective incident excitation light, and produce signals representative of the detected said autofluorescence emissions, or the detected said diffuse reflectance signals, or both;
at least one optical filter operable to filter the signals representative of the detected said autofluorescence emissions, or the detected said diffuse reflectance signals, or both;
a system controller in communication with the excitation light source, the at least one photodetector, and a non-transitory memory storing instructions, which instructions when executed cause the system controller to:
control the excitation light unit to sequentially produce the plurality of excitation lights;
receive and process the signals from the at least one photodetector for each sequential application of the plurality of excitation lights, and produce an image representative of the signals produced by each sequential application of the plurality of excitation lights; and
analyze the tissue sample using a plurality of the images to determine the presence of detrusor muscle tissue within the tissue sample.

2. The system of claim 1, wherein the plurality of excitation lights are in a UV wavelength range.

3. The system of claim 1, wherein the plurality of excitation lights are in the wavelength ranges of about 275-285 nm and about 345-371 nm and about 400-410 nm.

4. The system of claim 1, wherein the excitation light source includes a plurality of excitation light units, and each excitation light unit includes at least one UV LED.

5. The system of claim 1, wherein the autofluorescence emissions are in the visible region.

6. The system of claim 1, wherein the autofluorescence emissions are in the wavelength ranges of about 352-388 nm, 380-420 nm, 429-475 nm, 532-552 nm and 593-643 nm.

7. The system of claim 1, where the diffuse reflectance signals are representative of light collected in the visible region.

8. The system of claim 1, where the diffuse reflectance signals are representative of light collected at about 405 nm, 450 nm, and 620 nm.

9. The system of claim 1, wherein the instructions when executed cause the system controller to analyze the tissue sample using each image to identify the presence of diseased tissue within the tissue sample, the analysis including providing cellular or microstructural morphological information.

10. The system according to claim 1, wherein the tissue sample is produced by a TURBT procedure, a ERBT procedure, a biopsy, or a cystectomy.

11. The system of claim 1, wherein the system controller instructions includes at least one classifier that is trained using multispectral images.

12. The system of claim 11, wherein at least a first of the multispectral images is representative of the autofluorescence emissions and at least a second of the multispectral images is representative of the diffuse reflectance signals.

13. The system of claim 1, wherein the at least one of the biomolecules associated with the bladder wall tissue includes one or more of collagen, elastin. NADH, elastin, or hemoglobin.

14. A system for analyzing an in-vivo bladder tissue sample, comprising:
an excitation light source configured to selectively produce a plurality of excitation lights, each said excitation light centered on a wavelength distinct from the centered wavelength of the other said excitation lights, wherein at least one of the excitation light centered wavelengths is configured to produce autofluorescence emissions from one or more biomolecules associated with a bladder wall tissue, and a diffuse reflectance signals from the tissue sample, the system configured so that the plurality of excitation lights are incident to the tissue sample;
at least one photodetector configured to detect the autofluorescence emissions, or the diffuse reflectance signals, or both from the tissue sample as a result of the respective incident excitation light, and produce signals representative of the detected said autofluorescence emissions, or the detected said diffuse reflectance signals, or both;
at least one optical filter operable to filter the signals representative of the detected said autofluorescence emissions, or the detected said diffuse reflectance signals, or both;
a probe configured to be deployed within a bladder, the probe in communication with the excitation light source and the at least one photodetector, the probe configured to interrogate the bladder wall tissue with the plurality of excitation lights and to collect the autofluorescence emissions from one or more biomolecules associated with the bladder wall tissue and the diffuse reflectance signals from the bladder wall tissue; and
a system controller in communication with the excitation light source, the at least one photodetector, and a non-transitory memory storing instructions, which instructions when executed cause the system controller to:
control the excitation light unit to sequentially produce the plurality of excitation lights;
receive and process the signals from the at least one photodetector for each sequential application of the plurality of excitation lights, and produce an image representative of the signals produced by each sequential application of the plurality of excitation lights; and
analyze the tissue sample using a plurality of the images to determine the presence of detrusor muscle tissue within the tissue sample.

15. The system of claim 14, wherein the probe is configured to excise the bladder wall tissue sample.

16. A method of analyzing a bladder tissue sample, comprising:
sequentially interrogating the tissue sample with a plurality of excitation lights, each excitation light centered on a wavelength distinct from the centered wavelength of the other excitation lights, wherein at least one of the excitation light centered wavelengths is configured to produce autofluorescence emissions from one or more biomolecules associated with a bladder wall tissue, and a diffuse reflectance signals from the tissue sample;
using at least one photodetector to detect the autofluorescence emissions, or the diffuse reflectance signals, or both from the tissue sample, and to produce photodetector signals representative of the detected said autofluorescence emissions, or the detected said diffuse reflectance signals, or both;
filtering the light emitted or reflected from the tissue sample resulting from each said sequential interrogation of the tissue sample;
processing the photodetector signals for each sequential application of the plurality of excitation lights, including producing an image representative of the photodetector signals produced by each sequential application of the plurality of excitation lights; and
analyzing the tissue sample using each image to identify the presence of detrusor muscle tissue within the tissue sample.

17. The method of claim 16, wherein the bladder tissue sample is produced during a TURBT procedure.

18. The method of claim 16, wherein the bladder tissue sample is produced during a ERBT procedure.

19. The method of claim 16, wherein the bladder tissue sample is produced during a biopsy.

20. The method of claim 16, wherein the bladder tissue sample is produced during a cystectomy.

21. The method of claim 16, wherein the plurality of excitation lights are in a UV wavelength range.

22. The method of claim 16, wherein the plurality of excitation lights are in the wavelength ranges of about 275-285 nm and about 345-371 nm and about 400-410 nm.

23. The method of claim 16, wherein the autofluorescence emissions are in the visible region.

24. The method of claim 16, wherein the autofluorescence emissions are in the wavelength ranges of about 352-388 nm, 380-420 nm, 429-475 nm, 532-552 nm and 593-643 nm.

25. The method of claim 16, where the diffuse reflectance signals are representative of light collected in the visible region.

26. The method of claim 16, where the diffuse reflectance signals are representative of light collected at about 405 nm, 450 nm, and 620 nm.

27. The method of claim 16, wherein the analyzing step includes analyzing the bladder tissue sample using each image to identify the presence of diseased tissue within the bladder tissue sample, the analyzing including providing cellular or microstructural morphological information.

28. The method of claim 16, wherein the analyzing step utilizes one or more classifiers and at least one of the one or more classifiers is trained using a library of bladder tissue samples that have been multispectrally analyzed with and without a histological stain.

29. The method of claim 16, wherein the analyzing step includes empirically quantifying at least one of the biomolecules associated with a bladder wall tissue, the quantifying including determining a concentration of the at least one of the biomolecules associated with the bladder wall tissue.

30. The method of claim 29, wherein the at least one of the biomolecules associated with the bladder wall tissue includes one or more of collagen, elastin. NADH, elastin, or hemoglobin.

31. A method of analyzing an in-vivo bladder wall tissue sample, comprising:
inserting a probe into a subject's bladder;
sequentially interrogating the bladder wall tissue sample with a plurality of excitation lights emanating from the probe, each excitation light centered on a wavelength distinct from the centered wavelength of the other excitation lights, wherein at least one of the excitation light centered wavelengths is configured to produce autofluorescence emissions from one or more biomolecules associated with a bladder wall tissue, and a diffuse reflectance signals from the tissue sample;
using at least one photodetector to detect the autofluorescence emissions, or the diffuse reflectance signals, or both collected from the bladder wall tissue sample using the probe, and to produce photodetector signals representative of the detected said autofluorescence emissions, or the detected said diffuse reflectance signals, or both;
filtering the light emitted or reflected from the tissue sample resulting from each said sequential interrogation of the tissue sample;
processing the photodetector signals for each sequential application of the plurality of excitation lights, including producing an image representative of the photodetector signals produced by each sequential application of the plurality of excitation lights; and
analyzing the bladder wall tissue sample using each image to identify the presence of detrusor muscle tissue within the bladder wall tissue sample.
